(19) United States
(12) Patent Application Publication
(10) Pub. No.: US 2024/0390336 A1
(43) Pub. Date: Nov. 28, 2024

(54) Methods for Preventing Ectopic Brain Mineralization in Alzheimer's Disease and Dementias

(71) Applicants: Erming Tian, Little Rock, AR (US); Maurizio Zangari, Little Rock, AR (US)

(72) Inventors: Erming Tian, Little Rock, AR (US); Maurizio Zangari, Little Rock, AR (US)

(73) Assignee: Tzerma LLC, Little Rock, AR (US)

(21) Appl. No.: 18/796,446

(22) Filed: Aug. 7, 2024

Related U.S. Application Data
(63) Continuation of application No. PCT/US2023/062380, filed on Feb. 10, 2023.
(60) Provisional application No. 63/309,047, filed on Feb. 11, 2022.

(51) Int. Cl. A61K 31/429; A61K 31/662; A61P 25/28; C12N 5/0793

(52) U.S. Cl. CPC A61K 31/429; A61K 31/662; A61P 25/28; C12N 5/0619; C12N 2501/73; C12N 2501/999

(57) ABSTRACT

Provided herein are methods for preventing biomineralization in brain tissue or neuronal tissue in vivo or in vitro by inhibiting the deposition of hydroxyapatite therein. Also provided are methods for delaying the progression or onset of Alzheimer's disease in a subject and for preventing the development of Alzheimer's disease in a post-traumatic brain injury subject. Contacting the brain tissue or neuronal tissue with or administering to a subject the drugs Levamisole and/or Foscarnet and/or a bisphosphonate inhibits or decreases levels of alkaline phosphatase and calcium and counteracts alpha-glycerophosphate therein to inhibit the activation of spontaneous biomineralization within a brain tissue to decrease or prevent ectopic biomineralization.

### CROSS-REFERENCE TO RELATED APPLICATIONS

This non-provisional application claims the benefit of pending international application PCT/US2023/062380, filed Feb. 10, 2023, which claims the benefit of priority under 35 U.S.C. § 119 (e) of provisional application U.S. Ser. No. 63/309,047,filed Feb. 11, 2022, the entirety of both of which are hereby incorporated by reference.

### BACKGROUND OF THE INVENTION

### Field of the Invention

The invention relates to the fields of neurodegenerative diseases or disorders. More specifically, the present invention relates to the underlying biology of calcification in neurodegeneration and microangiopathy in the brain and to methods for the prevention of the onset of Alzheimer's Disease or related dementias or the progression of established disease.

### Description of the Related Art

Alzheimer's disease (AD) is the most common cause of dementia. In the United States, one in nine people aged 65 and older (11.3%) has Alzheimer's disease and Alzheimer's disease related dementia (ADRD) (1). Besides aging, military personnel and athletes may also face the long-term consequence of traumatic brain injury (TBI)-caused neurodegeneration that increases risk for developing ADRD (2).

Alzheimer's disease is a slowly progressive neurodegeneration that begins many years before symptoms emerge (3) and is characterized by noticeable memory, language, thinking, or behavioral symptoms that impair a person's ability to function in daily life. It is believed that, during Alzheimer's disease development, beta-amyloid (Aβ) accumulation occurs 15-20 years before the tandem pathology spread of the protein beta-amyloid (plaques, Aβ) outside neurons (4) and the twisted strands of the protein tau (tangles) inside neurons in the brain (5), expediting cognitive decline. These biomarkers are accompanied by the death of neurons and damage to brain tissue (6-8).

Over the past decade, the focus of drug discovery and development efforts for Alzheimer's disease has shifted toward disease-modifying therapies effective to improve or to at least slow the loss of memory and cognition and to maintain independent function (9). The main therapeutic targets are β-amyloid peptides, followed by inflammatory mediators/factors and tau proteins (10, 11). The leading approach is passive immunization of Aβ and tau (12, 13). Currently, the Food and Drug Administration (FDA) has approved six drugs for the treatment of Alzheimer's disease. Five of these drugs are designed to temporarily treat Alzheimer's symptoms, but do not change the underlying biology of Alzheimer's disease or alter the course of the disease. The sixth drug, aducanumab, is intended to reduce beta-amyloid in the brain but is not a cure for Alzheimer's disease (1, 14-16).

Still, a number of challenges hinder the efforts to bring new Alzheimer's disease drugs to the market including the complex etiology, slowly progressive nature of Alzheimer's disease, the high level of comorbidity occurring in the elderly population, and the blood-brain barrier. In addition to insoluble senile plaques and neurofibrillary tangles, the role of brain tissue calcification (mineralization) in the etiology of Alzheimer's disease is increasingly being recognized, but whether the ectopic mineral accumulation is a consequence of Alzheimer's disease-associated changes in the brain or is a primary cause of Alzheimer's disease is still unclear (17-20).

Recently, significant increase of alkaline phosphatase (ALP) in the brain and serum of Alzheimer's disease and traumatic brain injury (TBI) cases have been linked to the formation of neurotoxic tau protein via the dephosphorylation effect of alkaline phosphatase (21-23). Alkaline phosphatase plays a pivotal role in brain tissue calcification because it is one of the three imperative elements to trigger biomineralization, i.e., an innate biological ability of producing hydroxyapatite crystals (HAP). When alkaline phosphatase is concomitantly present with calcium and glycerophosphate (GP), any given somatic cell including neuronal cells can be activated to synthesize and disperse hydroxyapatite. It is known that aberrant brain tissue calcification is highly frequent in the aging population. It is estimated a minimum between 5 and 100 million single-domain biogenic crystals are produced and deposited per gram of the brain tissue (17). The amalgamation of hydroxyapatite and neurofilaments forms insoluble proteinaceous matrixes resistant to natural proteolytic degradation.

Thus, the prior art is deficient in a fundamental understanding of brain tissue calcification in the conformation of insoluble senile plaques (Aβ) and neurofibrillary tangles (tau) and, concomitantly, methods of prevention and treatment of Alzheimer's Disease and related dementias. Particularly, the prior art is deficient in methods to prevent ectopic biomineralization in neuronal tissues, thereby inhibiting or delaying the onset of Alzheimer's Disease. The present invention fulfills this long-standing need and desire in the art.

### SUMMARY OF THE INVENTION

The present invention is directed to a method for inhibiting activation of a spontaneous biomineralization within a brain tissue. In this method, the brain tissue is contacted with a plurality of compounds effective to inhibit a concomitant increase therein of alkaline phosphatase, alpha-glycerophosphate or an acyclic alkane (CₙH₂ₙ₊₂) phosphoester salt.

The present invention is further directed to a method for preventing ectopic biomineralization in a neuronal tissue. The method comprises inhibiting the deposition of hydroxyapatite within the neuronal tissue, thereby preventing ectopic mineralization therein.

The present invention is directed further to a method for inhibiting biomineralization in brain tissue in a subject having Alzheimer's disease. In the method an amount of at least one drug therapeutically effective to prevent hydroxyapatite deposition via innate biomineralization in the brain tissue, thereby inhibiting biomineralization is administered to the subject.

The present invention is directed further still to a method for delaying or inhibiting the progression of Alzheimer's disease in a subject in need thereof. In the method in brain tissue of the subject an activity of alkaline phosphatase is decreased or alpha-glycerophosphate is counteracted or a combination thereof.

The present invention is directed further still to a method for delaying onset or progression of Alzheimer's disease and/or related dementias subsequent to traumatic brain injury in a high-risk subject. In this method, a first biofluid sample is obtained from the high-risk subject and a second biofluid sample is obtained from a control subject. A level of alkaline phosphatase is measured in the first biofluid sample and a level of alkaline phosphatase is measured in the second biofluid sample and the level of alkaline phosphatase in the first biofluid sample is compared with the level of alkaline phosphatase in the second biofluid sample. A first drug is administered to the high-risk subject to inhibit biomineralization in brain tissue therein when the level of alkaline phosphatase in the first biofluid sample is substantially greater than the level of alkaline phosphatase in the second biofluid sample, thereby delaying the onset or the progression of Alzheimer's disease and/or related dementias subsequent to the traumatic brain injury in the high-risk subject.

The present invention is directed to another related method that further comprises monitoring the progression of the Alzheimer's disease and/or the related dementias in the high-risk subject after an onset thereof. In the method after an interval of time a second biofluid sample is obtained from the high-risk subject and the level of alkaline phosphatase is measured in the second biofluid sample. The level of alkaline phosphatase in the second biofluid sample from the high-risk subject is compared with the level of alkaline phosphatase in the first biofluid sample from the subject. The level of alkaline phosphatase in the second biofluid sample that is less than or equal to the level of alkaline phosphatase in the first biofluid sample indicates that said first drug is delaying progression of the Alzheimer's disease and/or the related dementias and a regimen of administering the first drug to the high-risk subject is maintained.

The present invention is directed to another related method that further comprises measuring a level of alpha-glycerophosphate or an acyclic alkane (CₙH₂ₙ₊₂) phosphoester salt in the first biofluid sample and in the second biofluid sample. The level of the alpha-glycerophosphate or the acyclic alkane (CₙH₂ₙ₊₂) phosphoester salt in the first biofluid sample is compared with the level thereof in the second biofluid sample. A second drug is administered to the high-risk subject when the level of the alpha-glycerophosphate or the acyclic alkane (CₙH₂ₙ₊₂) phosphoester salt in the first biofluid sample is substantially greater than the level of alpha-glycerophosphate or an acyclic alkane (CₙH₂ₙ₊₂) phosphoester salt in the first biofluid sample.

The present invention is directed further still to a method for diagnosing and treating an onset of Alzheimer's Disease in a subject after a traumatic brain injury thereto. In this method, a first biofluid sample is obtained from the subject after the traumatic brain injury and a level of alkaline phosphatase in the first biofluid sample is measured as a baseline level. After an interval of time, a second biofluid sample is obtained from the subject and a level of alkaline phosphatase in the second biofluid sample is measured. The baseline level of alkaline phosphatase is compared with the level of alkaline phosphatase in the second biofluid sample. The level of alkaline phosphatase in the second biofluid sample that is substantially greater than the baseline level of alkaline phosphatase is indicative of the onset of the Alzheimer's Disease and a therapeutic amount of a drug effective to inhibit an activity of the alkaline phosphatase in brain tissue is administered to the subject, thereby diagnosing and treating the onset of Alzheimer's disease in the subject.

The present invention is directed to a related method that further comprises repeating at intervals the diagnostic and treatment steps. The present invention is directed to another related method that further comprises administering to the subject another drug therapeutically effective to counteract alpha-glycerophosphate or an acyclic alkane (CₙH₂ₙ₊₂) phosphoester salt in the brain tissue or a pharmaceutically acceptable composition thereof.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1B are electron micrographs of the decalcified neurofibrillary tangle (NFT) matrix (FIG. 1A) and of the amyloid-β (Aβ) cylindrical ridge (white arrow) (FIG. 1B) from brain biopsies of individuals with Alzheimer's disease (bars=0.2 μm).

FIG. 2A-2B illustrate a co-culture model of competitive biomineralization. FIG. 2A is a diagram of Saos-2 cell line (human osteosarcoma). A provider of tissue nonspecific alkaline phosphatase (TNAP) was separately cultured with human adherent cell lines that do not express alkaline phosphatase in a 6-well plate contains minimal essential medium (MEMα; 1.8 mM calcium), 10% fetal bovine serum (FBS) and 2 millimolar (mM) of alpha-glycerophosphate (αGP). FIG. 2B shows that after a 7-day co-culture, lower chambers and upper inserts were stained with Alizarin Red to indicate biomineralization activity by the intensity of hydroxyapatite produced by Saos-2 or HeLa (cervical adenocarcinoma), HCN-2 (cortical neuron) or HS-5 (bone marrow stroma) cell lines. HeLa and HCN-2 cell lines showed competitively higher biomineralization activity to use up tissue nonspecific alkaline phosphatase supplied by Saos-2 cells, which only showed biomineralization activity when culture alone (inverted black triangle) or with HS-5 cell line that had weaker biomineralization activity in co-culture.

FIGS. 3A-3F are microscopic studies of ectopic calcification in brain tissues. FIG. 3A: H&E stained nondemented hippocampus. FIG. 3B: H&E stained Alzheimer's disease hippocampus shows senile plaques (open arrows) and neurofibrillary tangles (solid arrows) in Alzheimer's disease. FIG. 3C: Alizarin Red stained (ARS) nondemented hippocampus. FIGS. 3D-3F: ARS Alzheimer's disease hippocampus with dense accumulation of hydroxyapatite (dark) in extracellular space and the cytosol (open triangles) and somatodendritic compartment (solid triangles) of neurons. (600× magnification).

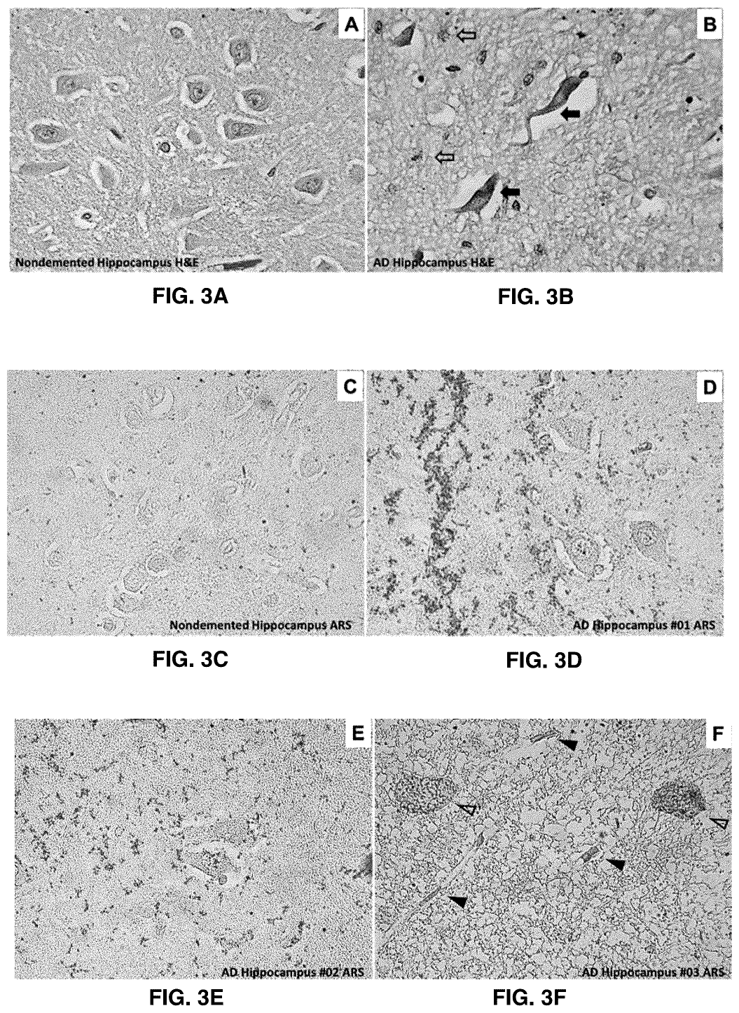

FIG. 4 is a microscopic study (top) AB of ectopic calcification in an Alzheimer's disease hippocampus stained with Alizarin Red, a 3D visualization (bottom) of mineralized electron-dense beta-amyloid plaque-like structures, mineralized blood vessels and unaffected vessels via tomographic reconstruction.

FIGS. 5A-5B show the human neuronal cell, HCN-2, exposed to MEMα supplemented with 10% FBS, αGP, and recombinant human tissue nonspecific alkaline phosphatase (TNAP). An antagonist to αGP (Foscarnet) (FIG. 5A) and an inhibitor of tissue nonspecific alkaline phosphatase (Levamisole) (FIG. 5B) were serially diluted and mixed with the medium to culture HCN-2 cells for 7 days. Alizarin Red staining of HAP indicates the biomineralization activity.

FIGS. 6A-6C show the biomineralization of human neuronal cell line, HCN-2. The cells were cultured in MEMα medium/10% FBS containing 2 mM of alpha-glycerophosphate and 1 IU/ml of human tissue nonspecific alkaline phosphatase for 24 hours. FIG. 6A: Bielschowsky's Silver stain showing neurofilaments (white arrows). FIG. 6B: Alizarin Red stained hydroxyapatite (dark particles). FIG. 6C: Superimposed neurofilaments and hydroxyapatite. The cell morphology was illustrated by optical phase-contrast. (600× magnification).

### DETAILED DESCRIPTION OF THE INVENTION

For convenience, before further description of the present invention, certain terms employed in the specification, examples and appended claims are collected herein. These definitions should be read in light of the remainder of the disclosure and understood as by a person of skill in the art. Unless defined otherwise, all technical and scientific terms used herein have the same meaning as commonly understood by a person of ordinary skill in the art.

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the term “including” is used herein to mean “including, but not limited to”. “Including” and “including, but not limited to” are used interchangeably.

As used herein, the term “about” refers to a numeric value, including, for example, whole numbers, fractions, and percentages, whether or not explicitly indicated. The term “about” generally refers to a range of numerical values (e.g., +5-10% of the recited value) that one of ordinary skill in the art would consider equivalent to the recited value (e.g., having the same function or result). In some instances, the term “about” may include numerical values that are rounded to the nearest significant figure.

As used herein, the ordinal adjectives “first” and “second”, unless otherwise specified are used to describe a common object, merely indicate that different instances of like objects are being referred to, and are not intended to imply that the objects so described must be in a given sequence, either temporally, spatially, in ranking, or in any other manner.

As used herein, the term “contacting” or “contact” refers to any suitable method of bringing a drug, an inhibitor, an antagonist, a compound or a pharmaceutical composition thereof into contact with a cell, for example, but not limited to, a neuronal cell. For in vivo applications, any known method of administration is suitable as described herein.

As used herein, the term “subject” refers to any human or non-human mammal recipient of the inhibitors, antagonists, compounds or pharmaceutical compositions thereof described herein.

As used herein, the term “high-risk” refers to a subject whose likelihood of developing Alzheimer's disease or related dementias at any age is significantly greater due to the elevated physiochemical profiles of alkaline phosphatase and glycerophosphate or a traumatic brain injury or to an increased possibility of suffering a traumatic brain injury over the general population due to, but not limited to, serving in the military and playing contact sports, for example, but not limited to, football, boxing, hockey and martial arts, particularly at a professional level.

As used herein, the term “pharmaceutically acceptable” refers to molecular entities and compositions that do not produce an adverse, allergic or other untoward reaction when administered to a subject, such as, for example, a human or non-human mammal, as appropriate. The preparation of a pharmaceutical composition that contains an inhibitor, an antagonist or compound is known to those of skill in the art in light of the present disclosure, as exemplified by Remington's Pharmaceutical Sciences, 18th Ed. Mack Printing Company, 1990, incorporated herein by reference.

As used herein, the term “therapeutically effective amount” refers to that amount of the drug inhibitor, antagonist and/or compound being administered to the subject sufficient to prevent progression of Alzheimer's disease or related dementia's and/or to inhibit or decrease or prevent biomineralization in neuronal cells.

In one embodiment of the present invention, there is provided a method for inhibiting activation of a spontaneous biomineralization within a brain tissue, comprising contacting the brain tissue with a plurality of compounds effective to inhibit a concomitant increase therein of alkaline phosphatase, alpha-glycerophosphate or an acyclic alkane (CₙH₂ₙ₊₂) phosphoester salt.

In this embodiment, the plurality of compounds may comprise Levamisol to inhibit the increase of alkaline phosphatase. In this embodiment, the plurality of compounds may comprise Foscarnet or a bisphosphonate to inhibit the increase of alpha-glycerophosphate or an acyclic alkane (CₙH₂ₙ₊₂) phosphoester salt. In addition, the contacting step may be performed in vitro or in vivo.

In another embodiment of the present invention, there is provided a method for preventing ectopic biomineralization in a neuronal tissue, comprising inhibiting the deposition of hydroxyapatite within the neuronal tissue, thereby preventing ectopic mineralization therein.

In one aspect of this embodiment, the inhibiting step may comprises contacting the neuronal tissue with a first drug effective to inactivate alkaline phosphatase therein. Particularly, the first drug is Levamisole or a pharmaceutically acceptable composition thereof. In another aspect of this embodiment, the inhibiting step may further comprise contacting the neuronal tissue with a second drug effective to counteract alpha-glycerophosphate therein. Particularly, the second drug is Foscarnet or a bisphosphonate or a pharmaceutically acceptable composition of each. In this further aspect, the second drug may contact the neuronal tissue concurrently with or sequentially to the first drug.

In this embodiment and aspects thereof, the inhibiting step may be performed in vivo or in vitro. In an aspect thereof, the inhibiting step may be performed in vivo on a subject with Alzheimer's disease or an Alzheimer's disease related dementia.

In yet another embodiment of the present invention, there is provided a method for inhibiting biomineralization in brain tissue in a subject having Alzheimer's disease, comprising administering to the subject an amount of at least one drug therapeutically effective to prevent hydroxyapatite deposition via innate biomineralization in the brain tissue, thereby inhibiting biomineralization. In this embodiment, the Alzheimer's disease may be an Alzheimer's disease related dementia.

In this embodiment, the drug may be Levamisole or a pharmaceutically acceptable composition thereof or Foscarnet or a pharmaceutically acceptable composition thereof or a bisphosphonate or a pharmaceutically acceptable composition thereof or a combination thereof. In one aspect Levamisole decreases a level of tissue nonspecific alkaline phosphatase in the brain tissue. In another aspect, Foscarnet or the bisphosphonate decreases a level of alpha-glycerophosphate or an acyclic alkane (CₙH₂ₙ₊₂) phosphoester salt in the brain tissue. In this embodiment and aspects thereof, the Levamisole the Foscarnet or the bisphosphonate may be administered sequentially or concurrently at least once.

In yet another embodiment of the present invention, there is provided a method for delaying or inhibiting the progression of Alzheimer's disease in a subject in need thereof, comprising decreasing an activity of alkaline phosphatase or counteracting alpha-glycerophosphate or a combination thereof in brain tissue of the subject. In this embodiment the Alzheimer's disease may be an Alzheimer's disease related dementia.

In one aspect of this embodiment, decreasing the activity of alkaline phosphatase may comprise administering to the subject a therapeutically effective amount of Levamisole or a pharmaceutically acceptable composition thereof. In another aspect, counteracting the alpha-glycerophosphate may comprise administering to the subject a therapeutically effective amount of Foscarnet, a therapeutically effective amount of a bisphosphonate or a pharmaceutically acceptable composition of each.

In yet another embodiment of the present invention, there is provided a method for delaying onset or progression of Alzheimer's disease and/or related dementias subsequent to traumatic brain injury in a high-risk subject in need thereof, comprising obtaining a first biofluid sample from the high-risk subject and a second biofluid sample from a control subject; measuring a level of alkaline phosphatase in the first biofluid sample and a level of alkaline phosphatase in the second biofluid sample; comparing the level of alkaline phosphatase in the first biofluid sample with the level of alkaline phosphatase in the second biofluid sample; and administering a first drug to the high-risk subject to inhibit biomineralization in brain tissue therein when the level of alkaline phosphatase in the first biofluid sample is substantially greater than the level of alkaline phosphatase in the second biofluid sample, thereby delaying the onset or the progression of Alzheimer's disease and/or related dementias subsequent to the traumatic brain injury in the high-risk subject. In this embodiment, the first drug may be Levamisole or a pharmaceutically acceptable composition thereof.

In a further embodiment, the method comprises monitoring the progression of the Alzheimer's disease and/or the related dementias in the high-risk subject after an onset thereof, comprising the steps of obtaining after an interval of time a second biofluid sample from the high-risk subject; measuring the level of alkaline phosphatase in the second biofluid sample; comparing the level of alkaline phosphatase in the second biofluid sample from the high-risk subject with the level of alkaline phosphatase in the first biofluid sample from the high-risk subject; wherein said level of alkaline phosphatase in the second biofluid sample that is less than or equal to the level of alkaline phosphatase in the first biofluid sample indicates that the first drug is delaying progression of the Alzheimer's disease and/or the related dementias; and maintaining a regimen of administering the first drug to the high-risk subject. In this further embodiment the first drug may be Levamisole or a pharmaceutically acceptable composition thereof.

In another further embodiment, the method comprises measuring a level of alpha-glycerophosphate or an acyclic alkane (CₙH₂ₙ₊₂) phosphoester salt in the first biofluid sample and in the second biofluid sample; comparing the level of the alpha-glycerophosphate or the acyclic alkane (CₙH₂ₙ₊₂) phosphoester salt in the first biofluid sample with the level thereof in the second biofluid sample; and administering a second drug to the high-risk subject when the level of the alpha-glycerophosphate or the acyclic alkane (CₙH₂ₙ₊₂) phosphoester salt in the first biofluid sample is substantially greater than the level of alpha-glycerophosphate or an acyclic alkane (CₙH₂ₙ₊₂) phosphoester salt in the first biofluid sample. In this further embodiment the second drug may be Foscarmet, a therapeutically effective amount of a bisphosphonate or a pharmaceutically acceptable composition of each. In all embodiments the biofluid may be peripheral blood serum or cerebrospinal fluid.

In yet another embodiment of the present invention, there is provided a method for diagnosing and treating an onset of Alzheimer's Disease in a subject after a traumatic brain injury thereto, comprising a) obtaining a first biofluid sample from the subject after the traumatic brain injury; b) measuring a level of alkaline phosphatase in the first biofluid sample as a baseline level; c) obtaining after an interval of time a second biofluid sample from the subject; d) measuring a level of alkaline phosphatase in the second biofluid sample; e) comparing the baseline level of alkaline phosphatase with the level of alkaline phosphatase in the second biofluid sample; wherein when the level of alkaline phosphatase in the second biofluid sample is substantially greater than the baseline level of alkaline phosphatase is indicative of the onset of the Alzheimer's Disease; and f) administering to the subject a therapeutic amount of a drug effective to inhibit an activity of the alkaline phosphatase in brain tissue, thereby diagnosing and treating the onset of Alzheimer's disease in the subject.

Further to this embodiment, the method comprises repeating at intervals steps b) to f). In both embodiments, the drug may be Levamisole or a pharmaceutically acceptable composition thereof.

In another further embodiment, the method comprises administering to the subject another drug therapeutically effective to counteract alpha-glycerophosphate or an acyclic alkane (CₙH₂ₙ₊₂) phosphoester salt in the brain tissue or a pharmaceutically acceptable composition thereof. In this further embodiment, the other drug is Foscarmet, a therapeutically effective amount of a bisphosphonate or a pharmaceutically acceptable composition of each. In all embodiments, the biofluid may be peripheral blood serum or cerebrospinal fluid.

Provided herein are methods for preventing biomineralization in a brain tissue or neuronal tissue or neuronal cells in vivo or in vitro to prevent the confirmation of senile plaques (Aβ) and neurofibrillary tangles (tau) via inhibition of hydroxyapatite deposition. It is demonstrated that insoluble senile plaques and neurofibrillary tangles in the AD brain are the aggregates of spatial beta-amyloid and tau proteins with hydroxyapatite crystals (HAP), which is produced by brain cells reacting to the presences of calcium, glycerophosphate and alkaline phosphatase in the microenvironment. The gender, race, traumatic brain injury, and unhealthy lifestyles, for example, due to fatty diet and gut microbiota, may contribute to the underlying condition of inappropriate biomineralization in the brain. Therefore, counterbalancing glycerophosphate and alkaline phosphatase, instead of targeting beta-amyloid and tau, may ultimately result in a clean-away of the neurotoxic proteins and prevent angiopathy.

In vitro, a drug, inhibitor, antagonist or other compound effective to inhibit brain tissue calcification is brought into contact with brain tissue or brain cells, for example, neuronal cells by methods known in the art. The compound is effective to inhibit or to decrease physiological levels of glycerophosphate (GP) and/or alkaline phosphatase (ALP) and/or calcium thereby decreasing or preventing activation of hydroxyapatite crystal (HAP) biosynthesis. For example, the concomitant increase of levels of any isoform of alkaline phosphatase, alpha-or beta-glycerophospate and calcium may activate the spontaneous biomineralization in brain tissue, such as, neuronal cells.

In vivo such prevention may be utilized to treat Alzheimer's disease or Alzheimer's disease related dementias (ADRD) or traumatic brain injury to delay, inhibit or halt the progression thereof. Generally, the drug, inhibitor, antagonist or other therapeutic compound effective to inhibit hydroxyapatite deposition and subsequent filament aggregates therewith may be utilized therapeutically or prophylactically when administered to a subject with Alzheimer's disease, traumatic brain injury or other related dementias. For example, the therapeutic and prophylactic compounds are effective for decreasing or inactivating alkaline phosphatase or counteracting or competitively inhibiting levels of α-glycerophosphate in the brain tissue or neuronal tissue. Alternatively, a pharmaceutical composition of one or more of the drugs, inhibitors, antagonists or other therapeutic and/or prophylactic compounds may be used.

Particularly, the drug Levamisole may be utilized therapeutically to decrease a level of or an activity of, inhibit or inactivate alkaline phosphatase and the drug Foscarnet or a bisphosphonate, for example, Etidronate, Clodronate, Tiludronate, Alendronate, Risedronate, Ibandronate, Pamidronate, or Zoledronic acid or a combination thereof, may be utilized to counteract, competitively inhibit or decrease a level of a glycerophosphate. The alkaline phosphatase may be human intestinal alkaline phosphatase (ALPI), human germ-cell alkaline phosphatase (ALPG), human placental alkaline phosphatase (ALPP), and/or human tissue nonspecific alkaline phosphatase (TNAP/ALPL). The glycerophosphate may be the isoforms alpha-glycerophosphate, beta-glycerophosphate, and/or acyclic alkane (CₙH₂ₙ₊₂) phosphoester salts.

Levamisole, Foscarnet and a bisphosphonate may be used singly or in combination. In combination the drugs may be administered one or more times sequentially or concurrently. One of ordinary skill in the art is well able to determine the dosage and dosing schedule depending on, but not limited to, the progression of Alzheimer's disease or related dementia or traumatic brain injury in the subject, the overall health of the subject, for example, whether or not comorbidities are present, and the age of the subject.

Also provided are methods for delaying the onset or progression of Alzheimer's Disease or Alzheimer's Disease related dementias in a subject which may or may not be subsequent to a traumatic brain injury. The elevated levels of one or more biomarkers or diagnostic biomarkers in the subject, for example, but not limited to, alkaline phosphatase and/or alpha-glycerophosphate or acyclic alkane (CₙH₂ₙ₊₂) phosphoester salt or other biomarker(s) associated with brain tissue calcification and Alzheimer's disease progression, are measured and quantified in a biological sample, for example, a biofluid (biological fluid) such as blood serum, preferably peripheral blood serum, or cerebrospinal fluid (CSF) by any suitable method known in the art. If the levels of the biomarker(s) are substantially or significantly elevated over those obtained from a control subject, the drugs Levamisole and/or Foscarnet and/or a bisphosphonate may be administered. This method may be used to monitor progression of Alzheimer's Disease and/or the related dimensions thereof by obtaining a second biofluid sample from the subject periodically after one or more intervals of time, measuring the diagnostic biomarker(s) and comparing to the second biofluid sample. A second measurement less than or equal to the first measurement indicates a delay in progression and that the regimen for the first drug is therapeutically or prophylactically effective.

Correspondingly, these methods may be used to diagnose and treat Alzheimer's Disease after a traumatic brain injury in the subject. A first biofluid sample from after the traumatic brain injury determines a baseline of the diagnostic biomarker, such as, but not limited to alkaline phosphatase. After an interval of time a second biofluid sample is used to determine another level of the diagnostic biomarker. A second level greater than, for example, substantially greater than, the first level is diagnostic of Alzheimer's Disease which may be treated by the therapeutic administration of a drug effective to inhibit, decrease, competitively inhibit, or counteract the diagnostic biomarker, for example, but not limited to, Levamisol and/or Foscarmet and/or a bisphosphonate, as described herein. Alternatively, the baseline may be established from a control, such as, from a subject with a traumatic brain injury, for example, from a soldier or from a professional athlete or amateur athlete in a contact sport.

The following examples are given for the purpose of illustrating various embodiments of the invention and are not meant to limit the present invention in any fashion.

### EXAMPLE 1

### Methods and Materials

### Mouse Model

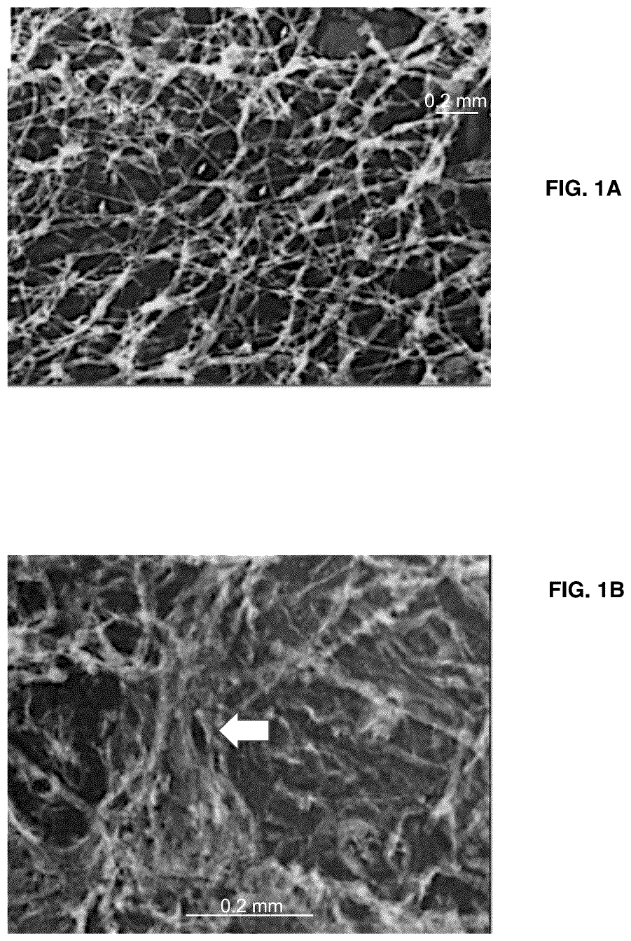

Two significant lesions have been identified in human Alzheimer's disease histopathology, i.e., extracellular senile plaques (SP) and intracellular neurofibrillary tangles (NFT) (24-28). Studies have shown that the architectures of senile plaques and neurofibrillary tangles are comprised of the irregular cylindrical ridges of amyloid-β (Aβ) and paired helical strands of hyperphosphorylated microtubule associated protein tau (tau) with large interfilamentous spaces >20 manometers (FIGS. 1A-1B). The commercially available Tau P301S (PS19) transgenic mouse model with the genomic mutations that instigate Alzheimer's disease (Jackson Laboratory; Bar Harbor, ME) are used. The PS19 mouse model harbors the T34 isoform of tau with one N-terminal insert and four microtubule binding repeats (1N4R) encoding the human tau P301S mutation. The neuron degeneration in hippocampus and ventricular dilatation (brain atrophy) can occur at eight months of age (29).

### Culture Media

Minimum essential medium alpha (MEMα) contains 1.8 mM of calcium supplemented with 10% fetal bovine serum (MEMα/FBS), alpha-glycerophosphate (αGP; 2 mM), and an alkaline phosphatase isozyme (human, bovine, sheep, or shrimp).

### Inhibitors

Levamisole or ((-)-(s)-2,3,5,6-tetrahydro-6-phenylimidazo (2,1-b) thiazole mono hydrochloride) is an inhibitor of tissue non-specific alkaline phosphatase. Foscarnet or phosphonoformic acid is a pyrophosphate analog DNA polymerase inhibitor. Bisphosphonates are organic analogs of pyrophosphate, i.e., that contain a P-C-P moiety that competitively inhibit the conjugation of alpha-glycerophosphate with alkaline phosphatase. These drugs cross the blood-brain barrier (30-31).

### EXAMPLE 2

### Biomineralization in Vitro

### Neuronal Cells Demonstrate a High Activity of Biomineralization to Produce Hydroxyapatite Crystals

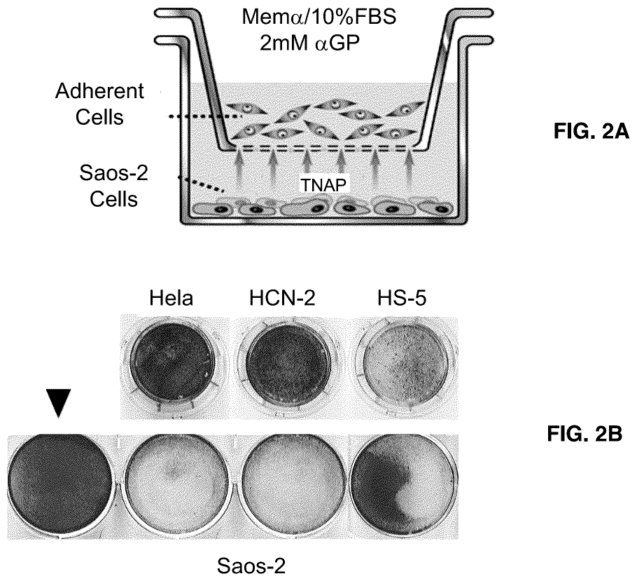

Biomineralization is an innate ability of any given somatic cell, including neuronal cells (32). In a cell-culture module that separates cell lines into different chambers (FIG. 2A), human cortical neuron cell line, HCN-2, and other adherent cell lines that do not express tissue nonspecific alkaline phosphatase were co-cultured with Saos-2 cell line (human osteosarcoma producing a high level of tissue nonspecific alkaline phosphatase) (33) in the minimum essential medium alpha containing 10% FBS and 2 mM of alpha-glycerophosphate. Within 7 days of co-culture, HCN-2 and HeLa (human cervical adenocarcinoma) cell lines demonstrated a high capability of biomineralization to synthesize hydroxyapatite (HAP) crystals as shown by Alizarin Red staining. In contrast, HS-5 (human bone marrow stroma cell line) was less active in biomineralization than HeLa and HCN-2 cell lines in the same co-culture condition (FIG. 2B). These assays demonstrate that when the microenvironment contains sufficient calcium and αGP, neuronal cells have a competitive advantage to “outsource” tissue nonspecific alkaline phosphatase from the donor cells (Saos-2) for biomineralization.

### Exclusive Recognition of HAP in Formalin Fixed Paraffin Embedded (FFPE) Autopsies

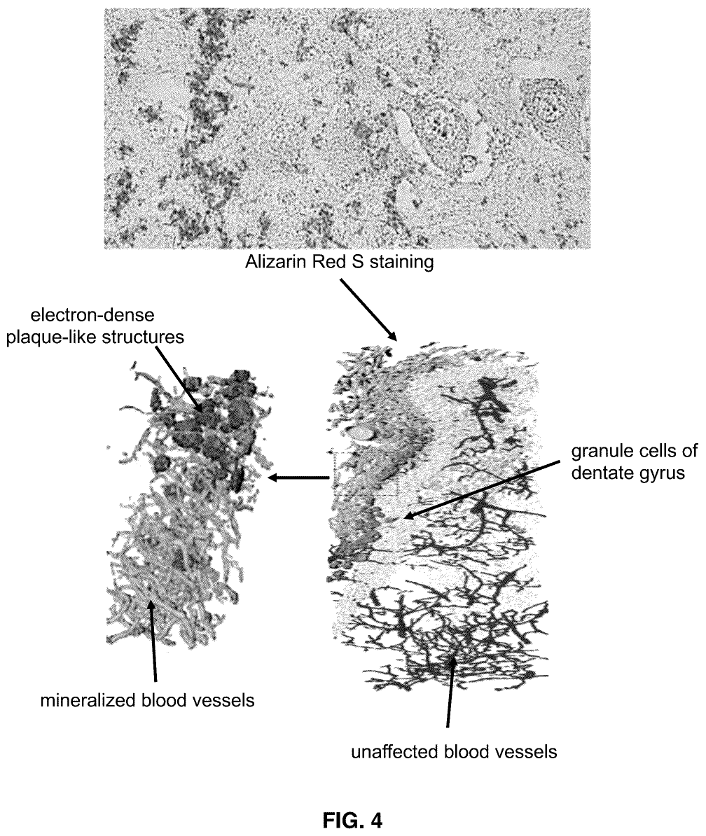

To corroborate the co-localization of hydroxyapatite and neurofibrillary proteins in the brain specimens, formalin-fixed, paraffin-embedded (FFPE) sections are stained with Alizarin Red and are examined. densely accumulated HAP in the interstitial space and the cytosol of the somatodendritic compartment of neurons in the Alzheimer's Disease hippocampus region, where senile plaques and neurofibrillary tangles were widely present (3A-3F). The colocalization of calcium-mineral with the classic plaques and tangles was corroborated by a recent study that correlated x-ray phase contrast tomography with histology to obtain 3D reconstructions of human hippocampal tissue affected by Alzheimer's Disease (34). A massive calcium deposition was associated with angiopathy at capillary level (FIG. 4) suggesting a broad impact of ectopic brain calcification in the etiology of Alzheimer's Disease.

### Biomineralization is a Controllable Mechanism

It is determined herein that the biomineralization reaction is dose-dependent on calcium, alpha-glycerophosphate and alkaline phosphatase. It is known that the physiological concentration of alkaline phosphatase in the brain is 1-2 international units per liter (IU/L), which is substantially lower than alkaline phosphatase in peripheral blood serum (44 to 147 IU/L)) (35, 36). Our titration assays indicated that biomineralization is dependent on the dosages of calcium, alpha-glycerophosphate, and alkaline phosphatase. The minimum dose of calcium, αGP, and TNAP that promptly triggers in vitro biomineralization reaction is 0.45 mM, 1.0 mM, and 100 IU/L, respectively (22). The reaction does not occur if any one of the three elements is deficient.

### The Quantitative Measurement of Alpha-glycerophosphate

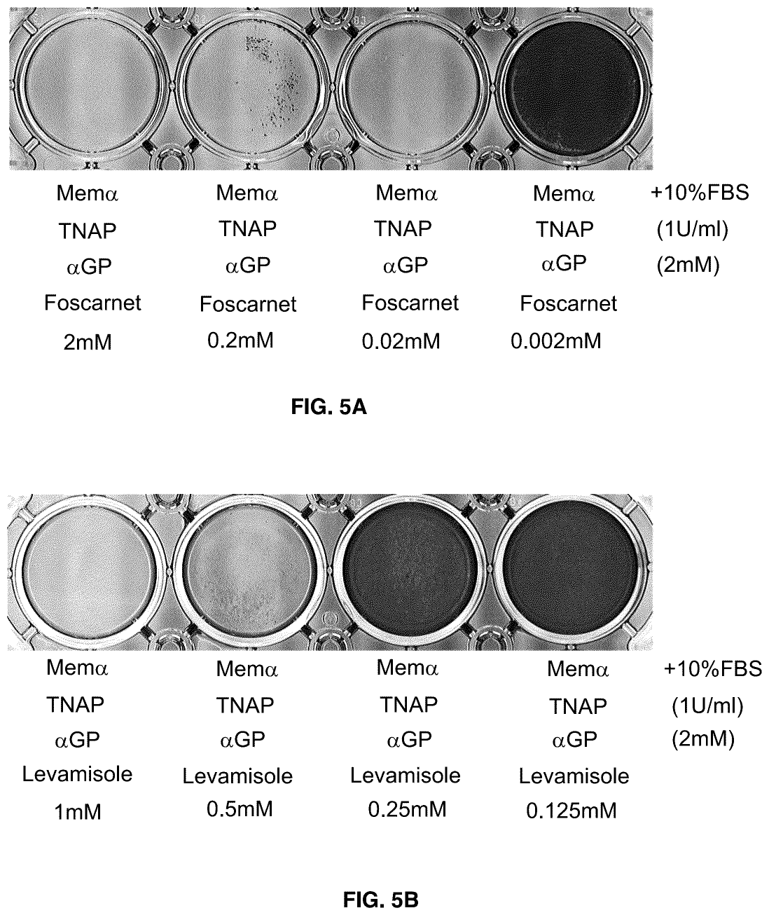

To measure the physiological glycerophosphate, a method to quantify this organic compound in peripheral blood serum was invented The method of quantifying alpha-glycerophosphate is disclosed in international publication WO 2021/076851, the entirety of which is hereby incorporated by reference. The results indicated that alpha-glycerophosphate (αGP) is the predominant isoform of GP and beta-glycerophosphate is undetectable. The physiological concentration of alpha-glycerophosphate varied substantially amid adult individuals with a mean value of 2.90 mM and standard deviation of 1.98 (median=2.42 mM, max=9.60 mM, and min=0.15 mM). The wide range of alpha-glycerophosphate in blood is likely attributed to personal diet, e.g., a fatty diet and gut microbiome, and physical activity because alpha-glycerophosphate is a metabolite derived from fat to energize mitochondria (37). Overall, 83% of tested samples contained alpha-glycerophosphate greater than 1.0 mM, which is sufficient to trigger biomineralization when calcium and alkaline phosphatase are concomitantly present. In vitro, a minimum 1.0 mM of alpha-glycerophosphate is required to trigger biomineralization reaction, and at least 0.02 mM of Foscarnet can competitively inhibit biomineralization (FIGS. 5A-5B). To cease biomineralization, it was discovered herein that Levamisole and Foscarnet demonstrate inhibitory effects to counterbalance alkaline posphatase and alpha-glycerophosphate triggering biomineralization (FIGS. 5A-5B). The proper dosages that adequately inhibit in vivo biomineralization activity are adjusted based on the concentration of alpha-glycerophosphate and alpha-glycerophosphate.

### The Co-localization of HAP with Aβ Plaques and Tau Tangles

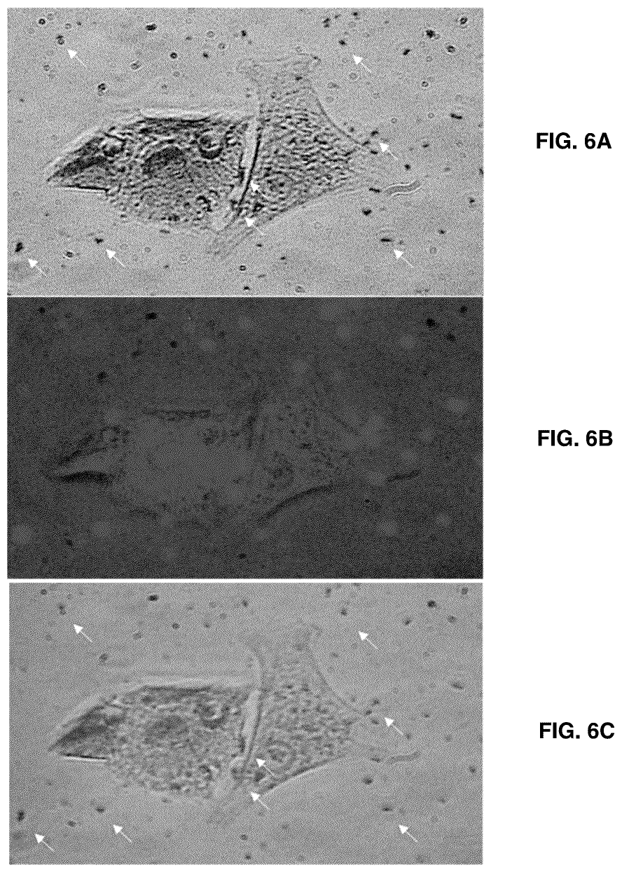

When HCN-2 cells (54) were exposed to MEMα supplemented with 10% FBS, alpha-glycerophosphate (2 mM), and human tissue nonspecific alkaline phosphatase (TNAP, 1 IU/ml), biomineralization is activated to produce and deposit HAP. The Alizarin red staining indicated that the aggregates of HAP with filaments secreted by HCN-2 cells could rapidly accumulate inside of the neuronal cells and in extracellular space within 24 hours (FIGS. 6A-6C).

### The Inhibition of Inate Biomineralization

Innate biomineralization is activated by three imperative elements, calcium, alpha-glycerophosphate, and alkaline phosphate in the microenvironment. When HCN-2 cells were exposed to MEMα/10% FBS supplemented with alpha-glycerophosphate (2 mM) and recombinant human tissue nonspecific alkaline phosphatase (1,000 U/L), hydroxyapatite produced by HCN-2 cells can be detected by Alizarin Red. The pharmaceutical compounds Foscarnet and Levamisole block biomineralization reactions. Essentially, at a ratio of 1:100 Foscarnet to alpha-glycerophosphate or 1 mM of Levamisole biomineralization is inhibited (FIGS. 5A-5B). Targeting either alpha-glycerophosphate or any isoform of alkaline phosphatase can competitively cease biomineralization. In vitro, each compound alone could entirely cease hydroxyapatite production by HCN-2 cells (FIGS. 5A-5B) indicating the potency of these compounds to prevent biomineralization.

### EXAMPLE 3

### Biomineralization In Vivo

### Tau Tangles Form via Elevated Biomineralization in Murine Neuronal Tissue

The levels of alpha-glycerophosphate and alkaline phosphatase in PS19 mice are regulated by injections of pharmaceutical grade disodium alpha-glycerophosphate (GLYCOPHOS; Fresenius Kabi USA) and recombinant human tissue nonspecific alkaline phosphatase (STRENSIQ; Alexion Pharmaceuticals) to mimic the in vivo phenotype of AD via accelerated biomineralization activity. In the experimental group, PS19 mice receive high dose of GLYCOPHOS and/or STRENSIQ to maintain a minimum 2 mM of αGP and 1,000 U/L of tissue nonspecific alkaline phosphatase throughout the test period.

### Inhibition of Alpha-glycerophosphate and Alkaline Phosphatase In Vivo

Foscarnet and Levamisole counterbalance alpha-glycerophosphate and alkaline phosphatase, respectively, in the experimental groups of PS19 mice with or without the injections of GLYCOPHOS and STRENSIQ. The behavioral tests on cognitive impairment and ectopic calcification in the brain are performed at the endpoints. To avoid discomfort, all drugs administrated to the mice are pharmaceutical agents approved by FDA. The tissue collection and preservation follow a humane euthanasia method and a standard FFPE protocol. The tissue sections are stained with hematoxylin and eosin (H&E) for histology and Alizarin Red staining is used to detect HAP crystallization.

The following references are cited herein.

1. 2022 Alzheimer's disease facts and figures. Alzheimer's Dement 18: 700-789.

2. Bryden et al. J Exp Neur 2019; 13:1-11, 2019.

3. Sperling et al. Alzheimer's Dement, 7 (3): 280-292, 2011.

4. Glenner G G, Wong C W. Biochem Biophys Res Commun. 120 (3): 885-890, 1984.

5. Grundke-Iqbal et al. PNAS 83 (13): 4913-3917, 1986.

6. Arnold et al. Cerebral Cortex, 1:103-116, 1991.

7. Arriagada et al. Neurology, 42:631-639, 1992.

8. Hanseeuw et al. JAMA Neurol. 76 (8): 915-924, 2019.

9. Salloway et al. Alzheimers Dement. 4 (2): 65-79, 2008.

10. Orr et al. Trends Pharmacol Sci. 38 (7): 637-48, 2017.

11. Lanoiselée et al. PLOS Med. 14 (3): e1002270, 2017.

12. Wisniewski T and Goni F. Neuron 2015; 85:1162-76.

13. Valera et al. Neurotherapeutics. 13:179-89, 2016.

14. Tolar M et al. Alzheimers Res Ther. 12 (1): 95, 2020.

15. Panza et al. Nat Rev Neurol. 15 (2): 73-88, 2019.

16. Congdon E E, Sigurdsson E M. Nat Rev Neurol. 14 (7): 399-415, 2018.

17. Kirschvink et al. Proc Natl Acad Sci USA. 89 (16): 7683-87, 1992.

18. Deng H et al. Ageing Res Rev. 22:20-38, 2015.

19. Saade et al. J Radiol Case Rep. 13 (8): 1-18, 2019.

20. Bos et al. Alzheimers Dement. 8 (5 Suppl): S104-11, 2012.

21. Deracinois et al. Subcell BioChem. 76:125-151, 2015.

22. Vardy et al. Neurodegener Dis. 9 (1): 31-7, 2012.

23. Díaz-Hernández et al. J Biol Chem. 285 (42): 32539-48, 2010.

24. Salloway et al. Alzheimers Dement. 4 (2): 65-79, 2008.

25. Orr et al. Trends Pharmacol Sci. 38 (7): 637-48, 2017.

26. Costandi M. et al Nature. 559 (7715): S16-S17, 2018.

27. Querfurth H W and LaFerla F M. NEJM. 362 (4): 329-44, 2010.

28. Ballard et al. Lancet. 377 (9770): 1019-31, 2011.

29. Jankowsky J L and Zheng H. Molecular Neurodegeneration 12:89, 2017.

30. Hengge et al. Antimic Agents & Chemo. 1993; 1010-14, 1993.

31. Deracinois et al. Sub-cellular Bioche. 76:125-51, 2015.

32. Tian et al. Int J Mol Sci. 21 (14): 4820, 2020.

33. Orimo H and Shimada T. Mol Cell Biochem. 315 (1-2): 51-60, 2008.

34. Töpperwien et al. Neuroimage. 210:116523, 2020.

35. Vardy et al. Neurodegener Dis. 9 (1): 31-7, 2012.

36. Deracinois et al. Subcell BioChem. 76:125-151, 2015.

37. Shen et al. Plant Cell. 18 (2): 422-41, 2006.

38. Querfurth H W and LaFerla F M. NEJM. 362 (4): 329-44, 2010.

39. Ballard et al. Lancet. 377 (9770): 1019-31, 2011.

40. Costandi M. Nature. 559 (7715): S16-S17, 2018.

41. Ruben et al. Brain Res. 602 (2): 164-79, 1993.

42. Forsberg et al. J Neurochem. 149 (4): 452-70, 2019.

43. Pincus M R and Abraham N Z. Interpreting laboratory results. In: McPherson R A, Pincus M R, eds. Henry's Clinical Diagnosis and Management by Laboratory Methods, 23rd ed. St Louis, MO: Elsevier, Chapter 8, 2017.

44. Godyń et al. Pharma Report 68 (1): 127-138, 2016.

45. Cummings et al. Alzheimer's & Dement 8 (1): e12295, 2022.

46. Tian et al. Int J Mol Sci. 21 (14): 4820, 2020.

47. Jankowsky J L, Zheng H. Molecular Neurodegeneration 12:89, 2017.

48. Astolfo et al. J Synchrotron Radiat. 23 (Pt 3): 813-819, 2016.

49. Viola K L, Klein W L. Acta Neuropathol. 129 (2): 183-206, 2015.

50. Selkoe D J. J Prev Alzheimers Dis. 7 (3): 140-141, 2020.

51. Kosik K S. Mol Neurobiol. 4:171-179, 1990.

52. Braak et al. J Neuropathol Exp Neurol. 70 (11): 960-969, 2011.

53. Wharton et al. Acta Neuropathol Commun. 4:11, 2016.

54. Perry et al. PNAS 84 (9): 3033-3036, 1987.

55. Sherman et al. J Mech Behav Biomed Mater. 52:22-50, 2015.

56. Giachelli C M. Am. J Pathol. 154 (3): 671-675, 1999.

57. Boskey A L. J Cell Biochem. 72:83-91, 1998.

58. Tsolaki et al. Acta Biomater. 143:38-144, 2022.

59. Kellett et al. Int J Mol Epidemiol Genet. 2 (2): 114-121, 2011.

60. Boccardi et al. Aging Clin Exp Res. 33 (3): 603-609, 2021.

61. Gonzalez et al. BMJ Open Gastro, 7, 2020.

62. Rodin et al. J Clini Endo & Metab. 68 (6): 1123-1127, 1989.

63. Arbib et al. Clin. Exp. Obstet. Gynecol. 48 (6): 1393-1399, 2021.

64. Sanfilippo et al. Br J Anaesth. 112 (2): 298-303, 2014.

65. Cadosch et al. J Bone Joint Surg Am. 92 (3): 645-653, 2010.

66. Pettigrew et al. J Biol Chem. 263 (1): 135-9, 1988.

67. Hayashi et al. J Biol Chem. 239:3098-3105, 1964.

68. Weiner S, Dove P M. Reviews in Mineralogy and Geochemistry. 54 (1): 1-29, 2003.

69. Knoll A H, Reviews in Mineralogy and Geochemistry. 54 (1): 329-50, 2003.

70. Kawasaki K, Weiss K M. J Exp Zool B Mol Dev Evol. 306 (3): 295-316, 2006.

71. Gregory et al. Anal Biochem. 329 (1): 77-84, 2004.

72. Moore E W. J Clin Invest. 49 (2): 318-334, 1970.

73. Ronnett et al. Neuroscience. 63 (4): 1081-1099, 1994.

74. Verlhac et al. Presse Med. 13;28 (6): 281, 1999.

What is claimed is:

1. A method for inhibiting activation of a spontaneous biomineralization within a brain tissue, comprising:
contacting the brain tissue with a plurality of compounds effective to inhibit a concomitant increase therein of alkaline phosphatase, alpha-glycerophosphate or an acyclic alkane (CₙH₂ₙ₊₂) phosphoester salt.

2. The method of claim 1, wherein the plurality of compounds comprises Levamisol to inhibit the increase of alkaline phosphatase.

3. The method of claim 1, wherein the plurality of compounds comprises Foscarnet or a bisphosphonate to inhibit the increase of alpha-glycerophosphate or beta-glycerophosphate or an acyclic alkane (CₙH₂ₙ₊₂) phosphoester salt.

4. The method of claim 1, wherein the contacting step is performed in vitro or in vivo.

5. A method for preventing ectopic biomineralization in a neuronal tissue, comprising:
inhibiting the deposition of hydroxyapatite within the neuronal tissue, thereby preventing ectopic mineralization therein.

6. The method of claim 5, wherein the inhibiting step comprises contacting the neuronal tissue with a first drug effective to inactivate alkaline phosphatase therein.

7. The method of claim 6, wherein the first drug is Levamisole or a pharmaceutically acceptable composition thereof.

8. The method of claim 6, wherein the inhibiting step further comprises contacting the neuronal tissue with a second drug effective to counteract alpha-glycerophosphate therein.

9. The method of claim 8, wherein the second drug is Foscarnet or a biphosphonate or a pharmaceutically acceptable composition of each.

10. The method of claim 8, wherein the second drug contacts the neuronal tissue concurrently with or sequentially to the first drug.

11. The method of claim 5, wherein the inhibiting step is performed in vivo or in vitro.

12. The method of claim 11, wherein the inhibiting step is performed in vivo on a subject with Alzheimer's disease or an Alzheimer's disease related dementia.

13. A method for delaying onset or progression of Alzheimer's disease and/or related dementias subsequent to traumatic brain injury in a high-risk subject in need thereof, comprising:
obtaining a first biofluid sample from the high-risk subject and a second biofluid sample from a control subject;
measuring a level of alkaline phosphatase in the first biofluid sample and a level of alkaline phosphatase in the second biofluid sample;
comparing the level of alkaline phosphatase in the first biofluid sample with the level of alkaline phosphatase in the second biofluid sample; and
administering a first drug to the subject to inhibit biomineralization in brain tissue therein when the level of alkaline phosphatase in the first biofluid sample is substantially greater than the level of alkaline phosphatase in the second biofluid sample, thereby delaying the onset or the progression of Alzheimer's disease and/or related dementias subsequent to the traumatic brain injury in the high-risk subject.

14. The method of claim 13, further comprising monitoring the progression of the Alzheimer's disease and/or the related dementias in the high-risk subject after an onset thereof, comprising the steps of:
obtaining after an interval of time a second biofluid sample from the high-risk subject;
measuring the level of alkaline phosphatase in the second biofluid sample;
comparing the level of alkaline phosphatase in the second biofluid sample from the high-risk subject with the level of alkaline phosphatase in the first biofluid sample from the high-risk subject; wherein said level of alkaline phosphatase in the second biofluid sample that is less than or equal to the level of alkaline phosphatase in the first biofluid sample indicates that said first drug is delaying progression of the Alzheimer's disease and/or the related dementias; and
maintaining a regimen of administering the first drug to the high-risk subject.

15. The method of claim 14, wherein the first drug is Levamisole or a pharmaceutically acceptable composition thereof.

16. The method of claim 13, further comprising:
measuring a level of alpha-glycerophosphate or an acyclic alkane (CₙH₂ₙ₊₂) phosphoester salt in the first biofluid sample and in the second biofluid sample;
comparing the level of the alpha-glycerophosphate or the acyclic alkane (CₙH₂ₙ₊₂) phosphoester salt in the first biofluid sample with the level thereof in the second biofluid sample; and
administering a second drug to the high-risk subject when the level of the alpha-glycerophosphate or the acyclic alkane (CₙH₂ₙ₊₂) phosphoester salt in the first biofluid sample is substantially greater than the level of alpha-glycerophosphate or an acyclic alkane (CₙH₂ₙ₊₂) phosphoester salt in the first biofluid sample.

17. The method of claim 16, wherein the second drug is Foscarmet, a therapeutically effective amount of a bisphosphonate or a pharmaceutically acceptable composition of each.

18. The method of claim 13, wherein the biofluid is peripheral blood serum or cerebrospinal fluid.

19. A method for diagnosing and treating an onset of Alzheimer's Disease in a subject after a traumatic brain injury thereto, comprising:
a) obtaining a first biofluid sample from the subject after the traumatic brain injury;
b) measuring a level of alkaline phosphatase in the first biofluid sample as a baseline level;
c) obtaining after an interval of time a second biofluid sample from the subject;
d) measuring a level of alkaline phosphatase in the second biofluid sample;
e) comparing the baseline level of alkaline phosphatase with the level of alkaline phosphatase in the second biofluid sample; wherein when the level of alkaline phosphatase in the second biofluid sample is substantially greater than the baseline level of alkaline phosphatase is indicative of the onset of the Alzheimer's Disease; and
f) administering to the subject a therapeutic amount of a drug effective to inhibit an activity of the alkaline phosphatase in brain tissue, thereby diagnosing and treating the onset of Alzheimer's disease in the subject.

20. The method of claim 19, comprising repeating at intervals steps b) to f).

21. The method of claim 19, wherein the drug is Levamisole or a pharmaceutically acceptable composition thereof.

22. The method of claim 19, further comprising administering to the subject another drug therapeutically effective to counteract alpha-glycerophosphate or an acyclic alkane (CₙH₂ₙ₊₂) phosphoester salt in the brain tissue or a pharmaceutically acceptable composition thereof.

23. The method of claim 22, wherein the other drug is Foscarmet, a therapeutically effective amount of a bisphosphonate or a pharmaceutically acceptable composition of each.

24. The method of claim 19, wherein the biofluid is peripheral blood serum or cerebrospinal fluid.
